(19) United States
(12) Patent Application Publication
(10) Pub. No.: US 2026/0191410 A1
(43) Pub. Date: Jul. 9, 2026

(54) OCT APPARATUS, STORAGE MEDIUM STORING OCT SIGNAL PROCESSING PROGRAM AND OCT SIGNAL PROCESSING METHOD

(71) Applicant: NIDEK CO.,LTD., Gamagori-shi (JP)

(72) Inventor: Ryosuke SHIBA, Gamagori-shi (JP)

(21) Appl. No.: 19/551,540

(22) Filed: Feb. 26, 2026

Related U.S. Application Data
(63) Continuation of application No. PCT/JP2024/032656, filed on Sep. 12, 2024.

(30) Foreign Application Priority Data
Sep. 29, 2023 (JP) 2023-169870

(51) Int. Cl. A61B 3/10; G06T 7/00; G06T 7/20

(52) U.S. Cl. CPC A61B 3/102; G06T 7/0016; G06T 7/20; G06T 2207/10101; G06T 2207/30041; G06T 2207/30104

(57) ABSTRACT

A control unit of the OCT apparatus performs a change-amount calculation step and a normalization step. In the change-amount calculation step, the control unit calculates an amount of change between a first complex OCT signal Cn and a second complex OCT signal Cn+1 acquired at different times at the same position on the biological tissue. In the normalization step, the control unit generates motion contrast data by normalizing the calculated amount of change based on a magnitude of the first complex OCT signal Cn and the second complex OCT signal Cn+1.

### CROSS REFERENCE TO RELATED APPLICATION

This application is a continuation application of International Patent Application No. PCT/JP2024/032656 filed on Sep. 12, 2024, which designated the U.S. and claims the benefit of priority from Japanese Patent Application No. 2023-169870 filed on Sep. 29, 2023. The entire disclosure of all of the above application is incorporated herein by reference.

### TECHNICAL FIELD

The present disclosure relates to an OCT apparatus, a storage medium storing an OCT signal processing program, and an OCT signal processing method that generate motion contrast data of a biological tissue by processing an OCT signal acquired based on the principle of optical coherence tomography (OCT).

### BACKGROUND

Conventionally, a technique for acquiring motion contrast data of a biological tissue (e.g., the fundus of an eye to be examined) based on the principle of OCT has been proposed. The motion contrast data are data obtained by processing a plurality of OCT signals acquired at different times from the same position on the biological tissue. In the motion contrast data, information on motion of the biological tissue (e.g., motion of blood flow in a blood vessel in the biological tissue) appears. Note that data indicating positions of blood vessels in the biological tissue (angiography data) and the like are examples of the motion contrast data.

As an OCT signal, a complex OCT signal expressed as a complex number is commonly used. The complex OCT signal can be expressed in polar form using an amplitude (absolute value) and a phase (argument).

Various methods for generating motion contrast data have been proposed. For example, in Patent Document 1 (JP 2015-131107 A), an OCT apparatus processes a plurality of OCT signals acquired at different times from the same position on a biological tissue, and generates first image data in which phase-difference information in the plurality of OCT signals is visualized. Further, the OCT apparatus processes the same plurality of OCT signals and generates second image data in which information including amplitudes of the plurality of OCT signals is visualized. By generating motion contrast data based on the information of the first image data and the information of the second image data, the OCT apparatus aims to compensate for disadvantages of both data with advantages of each other and to acquire a favorable image.

### SUMMARY

When complex OCT signals are acquired at different times for each of a plurality of positions in a biological tissue, both amplitude characteristics and phase characteristics differ among signals at positions where no blood vessel is present (hereinafter, referred to as “non-vessel positions”), signals at positions where a blood vessel is present (hereinafter, referred to as “vessel positions”), and noise. Therefore, by generating motion contrast data using both amplitude information and phase information, as compared with a case of using information of one of amplitude and phase, the quality of the generated data is more likely to be improved. Patent Document 1 also discloses a technique for generating motion contrast data using both phase information and amplitude information of an OCT signal. However, even when the method described in Patent Document 1 is used, favorable motion contrast data may not be generated in some cases.

A typical objective of the present disclosure is to provide an OCT apparatus, a storage medium storing an OCT signal processing program, and an OCT signal processing method that are capable of more appropriately acquiring motion contrast data of a biological tissue.

An OCT apparatus provided by a typical embodiment of the present disclosure includes: an OCT unit configured to detect an OCT signal based on reference light and measurement light irradiated onto a biological tissue of a subject; and a control unit configured to process the OCT signal to generate motion contrast data in the biological tissue. The control unit is further configured to perform: a change-amount calculation step of calculating an amount of change between a first complex OCT signal Cn and a second complex OCT signal Cn+1 that were acquired at different times at the same position on the biological tissue; and a normalization step of generating the motion contrast data by normalizing the calculated amount of change based on a magnitude of the first complex OCT signal Cn and the second complex OCT signal Cn+1.

A non-transitory, computer readable, storage medium storing an OCT signal processing program provided by a typical embodiment of the present disclosure is a storage medium storing an OCT signal processing program executed by an OCT signal processing apparatus that processes an OCT signal based on reference light and measurement light applied to a biological tissue of a subject. When the OCT signal processing program is executed by a control unit of the OCT signal processing apparatus, the program causes the OCT signal processing apparatus to perform: a change-amount calculation step of calculating an amount of change between a first complex OCT signal Cn and a second complex OCT signal Cn+1 that were acquired at different times at the same position on the biological tissue; and a normalization step of generating the motion contrast data by normalizing the calculated amount of change based on a magnitude of the first complex OCT signal Cn and the second complex OCT signal Cn+1.

An OCT signal processing method provided by a typical embodiment of the present disclosure is an OCT signal processing method implemented by an OCT signal processing apparatus that processes an CT signal obtained by reference light and measurement light irradiated onto a biological tissue of a subject. The OCT signal processing method includes: a change-amount calculation step of calculating an amount of change between a first complex OCT signal Cn and a second complex OCT signal Cn+1 that were acquired at different times at a same position on the biological tissue; and a normalization step of generating motion contrast data by normalizing the calculated amount of change based on a magnitude of the first complex OCT signal Cn and the second complex OCT signal Cn+1.

According to the OCT apparatus, the storage medium storing the OCT signal processing program, and the OCT signal processing methos according to the present disclosure, motion contrast data of a biological tissue can be obtained more appropriately.

### BRIEF DESCRIPTION OF THE DRAWINGS

FIG. 1 is a block diagram illustrating a schematic configuration of an OCT apparatus 1.

FIG. 2 is an explanatory diagram for describing an example of a method of acquiring an interference signal for a two-dimensional measurement region 55 on a biological tissue.

FIG. 3 is an explanatory diagram for describing an example of a method of acquiring interference signals of a plurality of frames at different times from the same scanning line.

FIG. 4 is a flowchart of OCT signal processing executed by the OCT apparatus 1 (OCT signal processing apparatus).

FIG. 5 is a diagram showing an example of a comparison result between a B-scan image before phase-difference correction and a B-scan image after phase-difference correction.

FIG. 6 is a diagram showing an example of a result of detecting layers and boundaries from a B-scan image.

FIG. 7 is a diagram showing, on a complex plane, an example of a relationship among a first complex OCT signal Cn, a second complex OCT signal Cn+1, and a complex difference between the two signals.

FIG. 8 is a diagram showing an example of an Enface image generated based on motion contrast data.

FIG. 9 is an explanatory diagram for describing differences in characteristics among a signal at a non-blood-vessel position, a signal at a blood-vessel position, and noise.

FIG. 10 is a graph comparing a value of a normalized complex difference with a value obtained by method Ar when a phase difference is varied while an amplitude value is fixed.

### DETAILED DESCRIPTION

The OCT apparatus exemplified in the present disclosure includes an OCT unit and a control unit. The OCT unit detects an OCT signal based on reference light and measurement light applied to a biological tissue of a subject. The control unit processes the OCT signal to generate motion contrast data in the biological tissue. The control unit executes a change-amount calculation step and a normalization step. In the change-amount calculation step, the control unit calculates an amount of change between a first complex OCT signal Cn and a second complex OCT signal Cn+1 acquired at different times from the same position on the biological tissue. In the normalization step, the control unit generates motion contrast data by normalizing the calculated amount of change based on a magnitude of the first complex OCT signal Cn and the second complex OCT signal Cn+1.

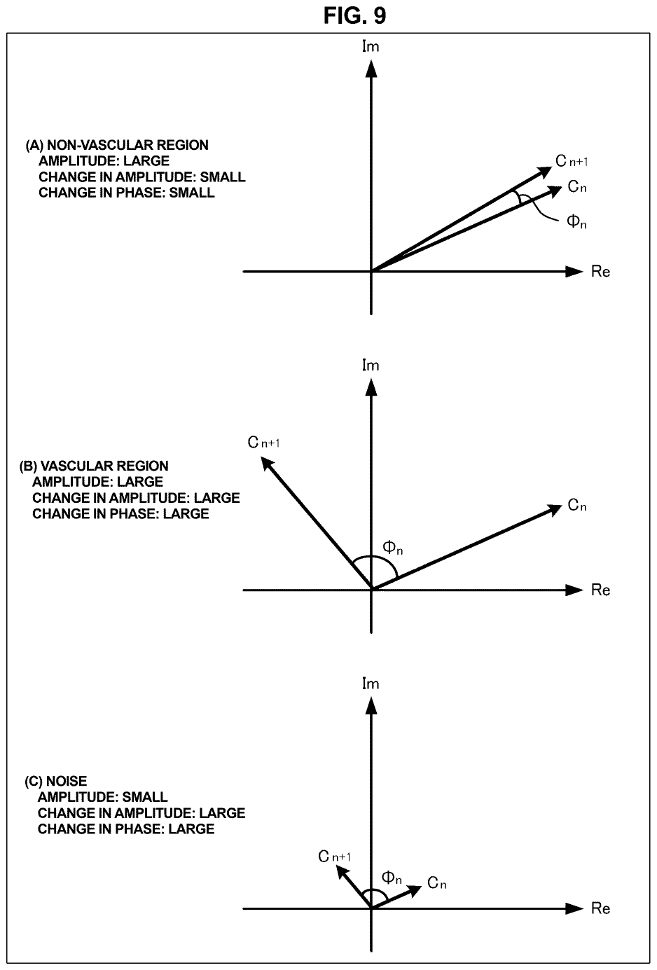

Here, the background of the technology according to the present disclosure will be described. When complex OCT signals are acquired at different times for each of a plurality of positions in a biological tissue, characteristics of a signal at a position where no blood vessel is present (hereinafter referred to as a “non-vascular position”), a signal at a position where a blood vessel is present (hereinafter referred to as a “vascular position”), and noise are different from each other. As illustrated in FIG. 9, when a plurality of complex OCT signals are acquired at a non-vascular position, the amplitude of each signal tends to be large, the change in amplitude between the plurality of signals tends to be small, and the change in phase between the plurality of signals also tends to be small. When a plurality of complex OCT signals are acquired at a vascular position, the amplitude of each signal, the change in amplitude between the plurality of signals, and the change in phase between the plurality of signals all tend to be large. Further, focusing on noise in each of a plurality of OCT signals acquired at different times from the same position, the amplitude of each noise component tends to be small, whereas both the change in amplitude between the plurality of noise components and the change in phase between the plurality of noise components tend to be large.

As described above, both amplitude characteristics and phase characteristics differ among the signal at a non-vascular position, the signal at a vascular position, and noise. Therefore, as compared with a case where information of one of amplitude and phase is used, quality of generated data tends to be improved by generating motion contrast data using information of both amplitude and phase. Patent Literature 1 also discloses a technique for generating motion contrast data using a difference (amount of change) between a first vector based on phase information and amplitude information of a first OCT signal and a second vector based on phase information and amplitude information of a second OCT signal. The difference between the two vectors in Patent Literature 1 is an example of an amount of change between two OCT signals.

However, an amount of change between a first complex OCT signal and a second complex OCT signal is susceptible to the influence of sensitivity of the complex OCT signal. Therefore, when an amount of change between a plurality of OCT signals is used without being normalized by signal magnitude, there is a case where, in a high-luminance region, a strong signal appears as an artifact in motion contrast data even when the subject actually does not move. For example, when a motion contrast image of a fundus is generated using an amount of change between a plurality of OCT signals, an artifact may appear in a high-luminance region such as a nerve fiber layer (NFL) and a retinal pigment epithelium (RPE) where luminance becomes high. In this case, it becomes difficult to distinguish an artifact in the high-luminance region from an actual blood-vessel region.

Therefore, as exemplified in Patent Literature 1, it is assumed that first motion contrast data generated based on an amount of change between a plurality of OCT signals is multiplied by second motion contrast data that does not depend on the magnitude of the OCT signals. In this case, as described above, the sensitivity of the first motion contrast data is high. On the other hand, the sensitivity of the second motion contrast data inevitably becomes low. Therefore, when the two pieces of data are multiplied, artifacts in a high-luminance region may decrease; however, the sensitivity decreases overall. As a result, the likelihood increases that a blood-vessel region that is actually continuous is interrupted.

In contrast, according to the technique exemplified in the present disclosure, first, in a change-amount calculation step, an amount of change between a first complex OCT signal Cn and a second complex OCT signal Cn+1 acquired at different times at the same position on a biological tissue is calculated. The amount of change calculated in the change-amount calculation step is an amount of change in which both phase information and amplitude information are taken into consideration. Next, in a normalization step, the amount of change between the first complex OCT signal Cn and the second complex OCT signal Cn+1 is normalized based on a magnitude of the two complex OCT signals, thereby generating motion contrast data. As a result, the amount of change calculated in the change-amount calculation step is normalized while influences of both phase information and amplitude information are maintained, and motion contrast data is generated. Therefore, the quality of the motion contrast data is improved as compared with a case where only one of amplitude and phase information is used. Further, unlike a case where first motion contrast data generated based on an amount of change between a plurality of OCT signals is multiplied by second motion contrast data that does not depend on the magnitude of the OCT signals, high sensitivity is also more likely to be maintained. As a result, better motion contrast data is generated.

It is also conceivable to first normalize each of the first complex OCT signal and the second complex OCT signal by the signal magnitude, and then calculate an amount of change of the values normalized by the magnitude. However, if each of the two complex OCT signals is normalized by the magnitude before calculating the amount of change, amplitude information is lost at that point, and motion contrast data based only on phase information is generated. Motion contrast data based only on phase information is susceptible to noise. In particular, when generating a motion-contrast still image, it is more desirable to use both amplitude information and phase information. In contrast, according to the technique of the present disclosure, motion contrast data is generated that uses both phase information and amplitude information and also has high sensitivity.

In the change-amount calculation step, the control unit may calculate, as the amount of change, the complex difference |Cn−Cn+1| between the first complex OCT signal Cn and the second complex OCT signal Cn+1. In this case, the amount of change between the first complex OCT signal and the second complex OCT signal is appropriately calculated in a state in which both phase information and amplitude information are taken into consideration.

In the normalization step, the control unit may normalize the amount of change by using, as the magnitude of the first complex OCT signal Cn and the first complex OCT signal Cn+1, a sum |Cn|+|Cn+1| of an absolute value |Cn| of the first complex OCT signal Cn and an absolute value |Cn+1| of the second complex OCT signal Cn+1. In this case, the control unit can appropriately normalize the amount of change calculated in the change-amount calculation step while maintaining influences of both phase information and amplitude information.

However, it is also possible to change a specific method of calculating the amount of change in the change-amount calculation step and a specific normalization method in the normalization step. For example, in the above-described example, the complex difference |Cn−Cn+1| is normalized such that the maximum value becomes 1 by dividing it by |Cn|+|Cn+1|. However, it is also possible to perform normalization such that the maximum value becomes 1 and the minimum value becomes 0. As an example, letting m=∥Cn|+|Cn+1∥ and M=|Cn|+|Cn+1|, normalization may be performed such that the maximum value becomes 1 and the minimum value becomes 0 by calculating (|Cn−Cn+1|−m)/(M−m). Further, a process of calculating at least one of a square of the complex OCT signal and a square of the complex difference, or the like, may be included in a process of generating the motion contrast data.

The control unit may execute a phase-difference correction step of correcting a phase difference of the first complex OCT signal Cn and a phase difference of the second complex OCT signal Cn+1. The control unit may execute the change-amount calculation step and the normalization step on the first complex OCT signal Cn whose phase difference has been corrected and the second complex OCT signal Cn+1 whose phase difference has been corrected. Ideally, the phase difference should be small in a region in which no blood vessel is present. However, in practice, even in a region in which no blood vessel is present, the phase difference between A-scan lines often increases within each B-scan image generated by each complex OCT signal due to biological fluctuations or the like. In this case, the quality of the generated motion contrast data deteriorates. Therefore, by correcting the phase difference of each of the two complex OCT signals, higher-quality motion contrast data is more easily generated.

Hereinafter, one of typical embodiments according to the present disclosure will be described. As an example, the OCT apparatus 1 of the present embodiment can process an OCT signal acquired using, as a subject, biological tissue of a fundus of an eye E to be examined. However, at least part of the techniques exemplified in the present disclosure can be applied also when processing an OCT signal of biological tissue other than the fundus in the eye E to be examined, or biological tissue other than the eye E to be examined (e.g., skin, a digestive organ, a brain, or a blood vessel (including cardiovascular vessels)). OCT data is data acquired based on the principle of optical coherence tomography (OCT).

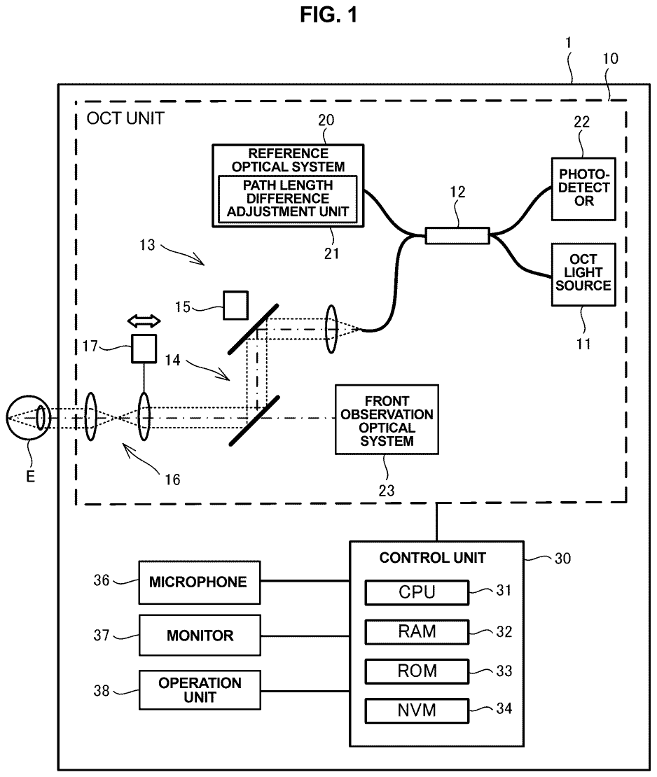

With reference to FIG. 1, a schematic configuration of the OCT apparatus 1 according to the present embodiment will be described. The OCT apparatus 1 includes an OCT unit 10 and a control unit 30. The OCT unit 10 includes an OCT light source 11, a coupler (optical splitter) 12, a measurement optical system 13, a reference optical system 20, a photodetector 22, and a front observation optical system 23.

The OCT light source 11 emits light (OCT light) for acquiring an OCT signal. The coupler 12 splits the OCT light emitted from the OCT light source 11 into measurement light and reference light. Further, the coupler 12 of the present embodiment multiplexes and causes interference between measurement light reflected by biological tissue of a subject (in the present embodiment, fundus tissue of an eye E to be examined) and reference light generated by the reference optical system 20. That is, the coupler 12 of the present embodiment serves also as a branching optical element that branches the OCT light into measurement light and reference light, and as a combining optical element that combines reflected light of the measurement light and the reference light. Note that it is also possible to change the configuration of at least one of the branching optical element and the combining optical element. For example, an element other than a coupler (e.g., a circulator, a beam splitter, etc.) may be used.

The measurement optical system 13 guides the measurement light split by the coupler 12 to the subject and returns the measurement light reflected by the subject to the coupler 12. The measurement optical system 13 includes a scanning unit 14, an irradiation optical system 16, and a focus adjustment unit 17. The scanning unit 14, by being driven by a driving unit 15, can scan (deflect) the measurement light in a two-dimensional direction intersecting the optical axis of the measurement light. In the present embodiment, two galvanometer mirrors capable of deflecting the measurement light in mutually different directions are used as the scanning unit 14. However, another device for deflecting light (e.g., at least one of a polygon mirror, a resonant scanner, an acousto-optic element, etc.) may be used as the scanning unit 14. The irradiation optical system 16 is provided downstream of the optical path relative to the scanning unit 14 (i.e., on the subject side) and irradiates tissue of the subject with the measurement light. The focus adjustment unit 17 adjusts the focus of the measurement light by moving an optical member (e.g., a lens) included in the irradiation optical system 16 in a direction along the optical axis of the measurement light.

The reference optical system 20 generates reference light and returns the reference light to the coupler 12. The reference optical system 20 of the present embodiment generates reference light by causing the reference light split by the coupler 12 to be reflected by a reflection optical system (e.g., a reference mirror). However, the configuration of the reference optical system 20 can also be modified. For example, the reference optical system 20 may transmit light incident from the coupler 12 without reflecting the light, and return the transmitted light to the coupler 12. The reference optical system 20 includes an optical path length difference adjustment unit 21 that changes an optical path length difference between the measurement light and the reference light. In the present embodiment, the optical path length difference is changed by moving the reference mirror in the optical axis direction. Note that a configuration for changing the optical path length difference may be provided in the optical path of the measurement optical system 13.

The photodetector 22 detects an interference signal by receiving interference light between the measurement light and the reference light generated by the coupler 12. In the present embodiment, the principle of Fourier-domain OCT is employed. In Fourier-domain OCT, the spectral intensity of interference light (a spectral interference signal) is detected by the photodetector 22, and a complex OCT signal is acquired by Fourier transform on the spectral intensity data. As an example of Fourier-domain OCT, Spectral-domain OCT (SD-OCT), Swept-source OCT (SS-OCT), and the like can be employed. Further, for example, Time-domain OCT (TD-OCT) and the like can also be employed.

In the present embodiment, SD-OCT is employed. In the case of SD-OCT, for example, a low-coherence light source (broadband light source) is used as the OCT light source 11, and a spectroscopic optical system (spectrometer) that disperses the interference light into respective frequency components (respective wavelength components) is provided in the vicinity of the photodetector 22 in the optical path of the interference light. In the case of SS-OCT, for example, a wavelength-swept light source (tunable light source) that temporally changes the emission wavelength at high speed is used as the OCT light source 11. In this case, the OCT light source 11 may include a light source, a fiber ring resonator, and a wavelength-selective filter. Examples of the wavelength-selective filter include a filter combining a diffraction grating and a polygon mirror, and a filter using a Fabry-Perot etalon.

In the present embodiment, three-dimensional OCT data (for example, a three-dimensional tomographic image) are acquired by scanning the spot of the measurement light within a two-dimensional measurement region by the scanning unit 14. However, the principle for acquiring the three-dimensional OCT data may be changed. For example, three-dimensional OCT data may be acquired based on the principle of line-field OCT (hereinafter referred to as “LF-OCT”). In LF-OCT, the measurement light is simultaneously applied to an irradiation line extending in a one-dimensional direction in tissue, and interference light between reflected light of the measurement light and the reference light is received by a one-dimensional photodetector (for example, a line sensor) or a two-dimensional photodetector. Three-dimensional OCT data are acquired by scanning the measurement light in a direction intersecting the irradiation line within the two-dimensional measurement region. Further, three-dimensional OCT data may be acquired based on the principle of full-field OCT (hereinafter referred to as “FF-OCT”). In FF-OCT, the measurement light is applied to a two-dimensional measurement region on tissue, and interference light between reflected light of the measurement light and the reference light is received by a two-dimensional photodetector. In this case, the OCT apparatus 1 may not include the scanning unit 14.

A front observation optical system 23 is provided to capture, in real time, a front observation image of biological tissue of a subject (in the present embodiment, the fundus of an eye E to be examined). In the present embodiment, the front observation image refers to a two-dimensional image obtained when tissue is viewed from a direction (front direction) along the optical axis of the OCT measurement light. In the present embodiment, a scanning laser ophthalmoscope (SLO) is employed as the front observation optical system 23. However, as the configuration of the front observation optical system 23, a configuration other than an SLO (for example, an infrared camera that captures a front image by collectively irradiating infrared light over a two-dimensional imaging range) may be employed.

The OCT apparatus 1 can acquire (generate), based on the acquired three-dimensional OCT data, an Enface image that is a two-dimensional front image obtained when tissue is viewed from a direction (front direction) along the optical axis of the measurement light. When an Enface image is acquired in real time, the acquired Enface image can also be used as the above-described front observation image. In this case, the front observation optical system 23 can be omitted. The data of the Enface image may be, for example, integrated image data in which luminance values are integrated in the depth direction (Z direction) at each position in the XY direction, an integrated value of spectral data at each position in the XY direction, luminance data at each position in the XY direction at a certain depth, luminance data at each position in the XY direction in any layer of the retina (for example, a superficial retinal layer), or the like. Further, the OCT apparatus 1 of the present embodiment can also generate an Enface image from motion contrast data. The motion contrast data are data obtained by processing a plurality of OCT signals acquired from the same position on biological tissue at different times. In the motion contrast data, information on movement of biological tissue (for example, movement of blood flow within a blood vessel in the biological tissue) appears. In the present embodiment, by generating an Enface image of a specific layer based on the motion contrast data, an angiographic image (vascular image), which is an image indicating a vessel position included in the specific layer, is generated.

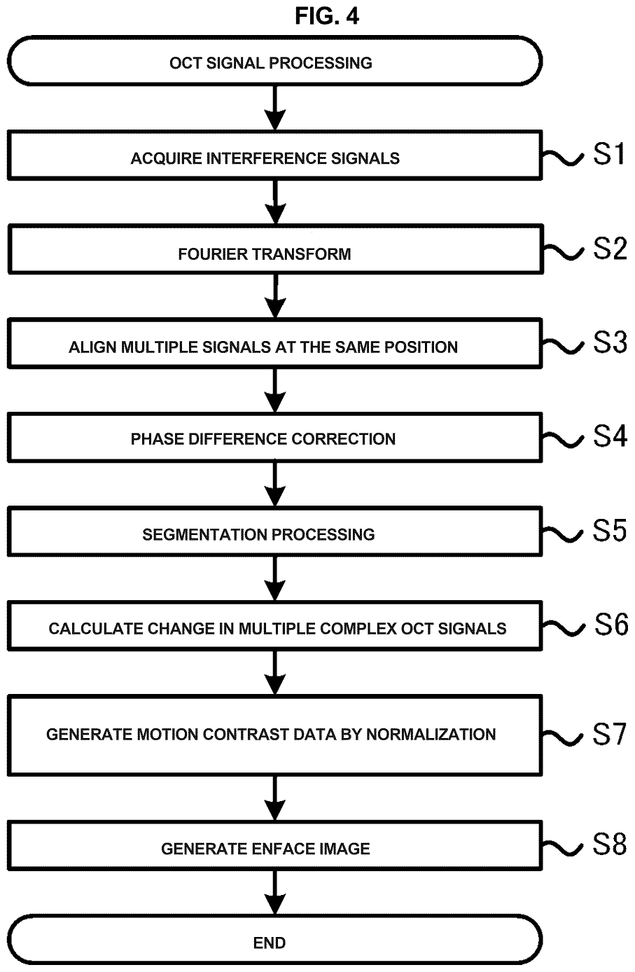

A control unit 30 is responsible for various controls of the OCT apparatus 1. The control unit 30 includes a CPU 31, a RAM 32, a ROM 33, and a non-volatile memory (NVM) 34. The CPU 31 is a controller including at least one processor and at least one memory. The CPU 31 is configured to perform various controls. The RAM 32 temporarily stores various types of information. The ROM 33 stores programs to be executed by the CPU 31 and various initial values, and the like. The NVM 34 is a non-transitory storage medium capable of retaining stored contents even when power supply is cut off. An OCT signal processing program for executing OCT signal processing (see FIG. 4) described later may be stored in the NVM 34.

In the present disclosure, the term “processor” may refer to a single hardware processor or several hardware processors that are configured to execute computer program code (i.e., one or more instructions of a program) included in a program. In other words, a processor may be one or more programmable hardware devices. For instance, a processor may be a general-purpose or embedded processor and include, but not necessarily limited to, CPU (a Central Processing Circuit), a microprocessor, GPU (a Graphics Processing Unit), and DFP (a Data Flow Processor).

The term “memory” in the present disclosure is a non-transitory, tangible storage medium, and may refer to a single or several hardware memory configured to store computer program code (i.e., one or more instructions of a program) and/or data accessible by a processor. A memory may be implemented using any suitable memory technology, such as static random-access memory (SRAM), synchronous dynamic RAM (SDRAM), nonvolatile/Flash-type memory, or any other type of memory. Computer program code constituting a program may be stored on the memory and, when executed by a processor, cause SUBJECT MATTER to perform the above-described various functions.

A microphone 36, a monitor 37, and an operation unit 38 are connected to the control unit 30. The microphone 36 inputs sound. The monitor 37 is an example of a display unit that displays various images. The operation unit 38 is operated by a user in order for the user to input various operation instructions to the OCT apparatus 1. As the operation unit 38, various devices such as, for example, a mouse, a keyboard, a touch panel, and a foot switch can be used. Note that various operation instructions may be input to the OCT apparatus 1 by inputting sound to the microphone 36. In this case, the CPU 31 may determine the type of the operation instruction by performing speech recognition processing on the input sound.

In the present embodiment, an integrated OCT apparatus 1 in which the OCT unit 10 and the control unit 30 are incorporated in a single housing is exemplified. However, it goes without saying that the OCT apparatus 1 may include a plurality of apparatuses having different housings. For example, the OCT apparatus 1 may include an optical apparatus incorporating the OCT unit 10, and a PC connected to the optical apparatus by wired or wireless connection. In this case, a control unit included in the optical apparatus and a control unit of the PC may both function as the control unit 30 of the OCT apparatus 1. Further, the OCT apparatus 1 of the present embodiment also serves as the OCT unit 10 that acquires an interference signal for biological tissue and an OCT signal processing apparatus that processes the acquired interference signal or OCT signal. However, an OCT signal processing apparatus may be used separately from the OCT apparatus 1 that acquires an interference signal. In this case, the OCT signal processing apparatus may acquire an interference signal or OCT signal for biological tissue acquired by the OCT apparatus 1 via wired communication, wireless communication, a network, a removable storage device, or the like, and may generate motion contrast data by processing the acquired signal. In this case, OCT signal processing described later may be executed by a control unit of the OCT signal processing apparatus.

### <Acquisition of Interference Signal>

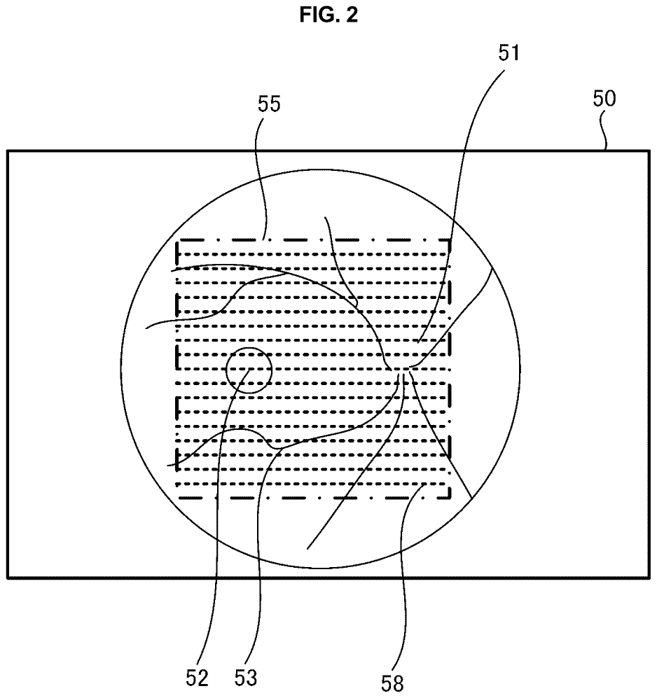

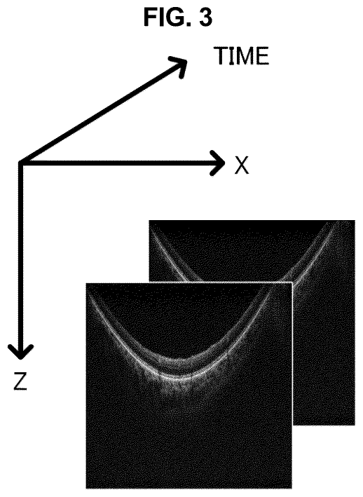

With reference to FIGS. 2 and 3, an example of a method by which the OCT apparatus 1 acquires an interference signal for biological tissue will be described. First, the CPU 31 starts capturing a two-dimensional en-face image of biological tissue to be subjected to acquisition of an interference signal (in the present embodiment, the fundus of the subject's eye E) by controlling the front observation optical system 23. In the example shown in FIG. 2, an optic disc (hereinafter also referred to as a “disc”) 51, a macula 52, and fundus blood vessels 53 of the subject's eye E are captured in the two-dimensional en-face image 50. The two-dimensional en-face image 50 is repeatedly and intermittently captured and displayed on the monitor 37 as a moving image.

The CPU 31 acquires an interference signal for a measurement region 55 on the biological tissue upon generation of a trigger signal that starts acquisition of the interference signal. In the present embodiment, the CPU 31 controls driving of the scanning unit 14 by the drive unit 15 and causes the spot of the measurement light to scan within the two-dimensional measurement region 55, thereby acquiring an interference signal for the measurement region 55. As an example, in the present embodiment, as shown in FIG. 3, a plurality of linear scanning lines (scan lines) 58 for scanning the spot are set in the measurement region 55 at equal intervals, and the spot of the measurement light is scanned on each scanning line 58, thereby acquiring an interference signal for the two-dimensional measurement region 55.

More specifically, the CPU 31 acquires at least two frames of interference signals at different times from the same position on the biological tissue (in the example shown in FIG. 3, the same scanning line 58). In the example shown in FIG. 3, the CPU 31 first acquires an interference signal detected by the light-receiving element 22 by scanning the measurement light on the first scanning line 58 among the plurality of scanning lines 58. Hereinafter, the direction in which the scanning lines 58 extend is defined as the X direction. One scan of the measurement light in the X direction on each scanning line 58 is referred to as a “B-scan.” A two-dimensional image generated by a B-scan is referred to as a “B-scan image.” In a B-scan image, each of a plurality of pixel columns extending in a direction along the optical axis of the measurement light is referred to as an “A-scan image.” Hereinafter, one frame of the interference signal will be described as an interference signal acquired by one B-scan. Further, the Z direction is defined as a direction along the optical axis of the measurement light. The Y direction is a direction intersecting both the X direction and the Z direction (in the present embodiment, a direction intersecting perpendicularly).

When a first B-scan for the first scanning line 58 is completed, the CPU 31 acquires an interference signal of a second frame by performing a second B-scan on the first scanning line 58. As a result, as shown in FIG. 3, two frames of interference signals are acquired from the first scanning line 58 at different times. Note that the CPU 31 can also acquire interference signals of three or more frames from the same position (e.g., on the same scanning line 58).

Note that, if it is possible to acquire interference signals of a plurality of frames from the same scanning line 58 at different times by scanning the measurement light once on the scanning line 58, it is unnecessary to scan the measurement light a plurality of times on the same scanning line 58. For example, when two measurement lights whose optical axes are shifted by a predetermined interval are scanned at one time, it is unnecessary to scan the measurement light a plurality of times on the same scanning line 58.

When acquisition of interference signals of a plurality of frames from the first scanning line 58 is completed, the CPU 31 moves the position at which a B-scan is performed in parallel in the Y direction, and executes processing for acquiring interference signals of a plurality of frames from the second scanning line 58. By performing the above processing for each of a plurality of scanning lines 58, interference signals for the two-dimensional measurement region 55 are acquired. Note that the direction of the first B-scan and the direction of the second B-scan on the same scanning line 58 may be reversed, or a plurality of B-scans may be repeated in the same direction.

### <October Signal Processing>

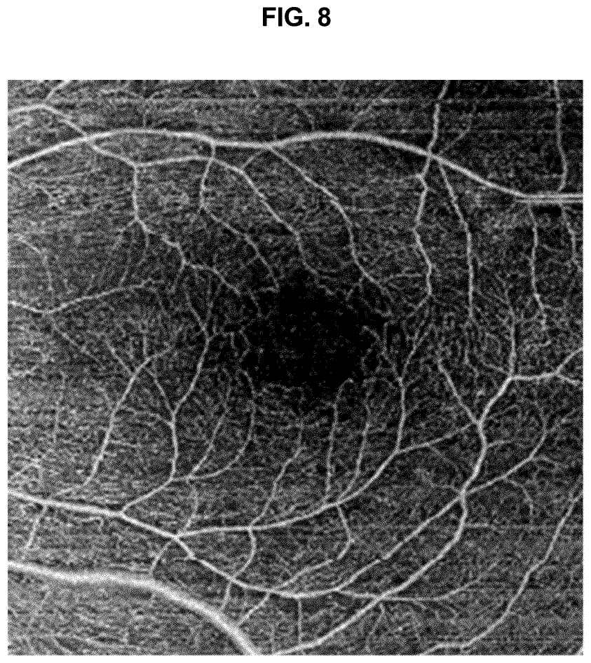

With reference to FIGS. 4 to 8, OCT signal processing executed by an OCT signal processing apparatus (in the present embodiment, the OCT apparatus 1) will be described. In the OCT signal processing, motion contrast data of a biological tissue is generated by processing interference signals acquired from the biological tissue by the OCT unit 10 of the OCT apparatus 1. The CPU 31 of the OCT apparatus 1 executes the OCT signal processing shown in FIG. 4 in accordance with an OCT signal processing program stored in the NVM 34.

As shown in FIG. 4, the CPU 31 acquires an interference signal acquired from a biological tissue by the OCT unit 10 (S1). The CPU 31 acquires a complex OCT signal by performing Fourier transform on the interference signal acquired in S1 (S2). As described above, in the present embodiment, a first interference signal In and a second interference signal In+1 are acquired at different times from each of a plurality of positions on a biological tissue. By Fourier-transforming the first interference signal In, a first complex OCT signal Cn (amplitude An, phase θn) is acquired. By Fourier-transforming the second interference signal In+1, a second complex OCT signal Cn+1 (amplitude An+1, phase θn+1) is acquired.

The CPU 31 performs alignment (image registration) between the first complex OCT signal Cn and the second complex OCT signal Cn+1 acquired from the same position on the biological tissue at different times (S3). In the present embodiment, the CPU 31 performs the alignment by arranging and positioning a plurality of images of the same scene (a B-scan image generated by the first complex OCT signal Cn and a B-scan image generated by the second complex OCT signal Cn+1) in correspondence with each other. In order to appropriately generate motion contrast data indicating tissue motion (in the present embodiment, blood flow), it is necessary to compare signals acquired at different times from the same position. However, due to motion of the biological tissue during imaging and the like, there may be a case where the position at which the first complex OCT signal Cn is acquired and the position at which the second complex OCT signal Cn+1 is acquired are misaligned. Therefore, the CPU 31 improves the quality of the motion contrast data by performing alignment between the first complex OCT signal Cn and the second complex OCT signal Cn+1.

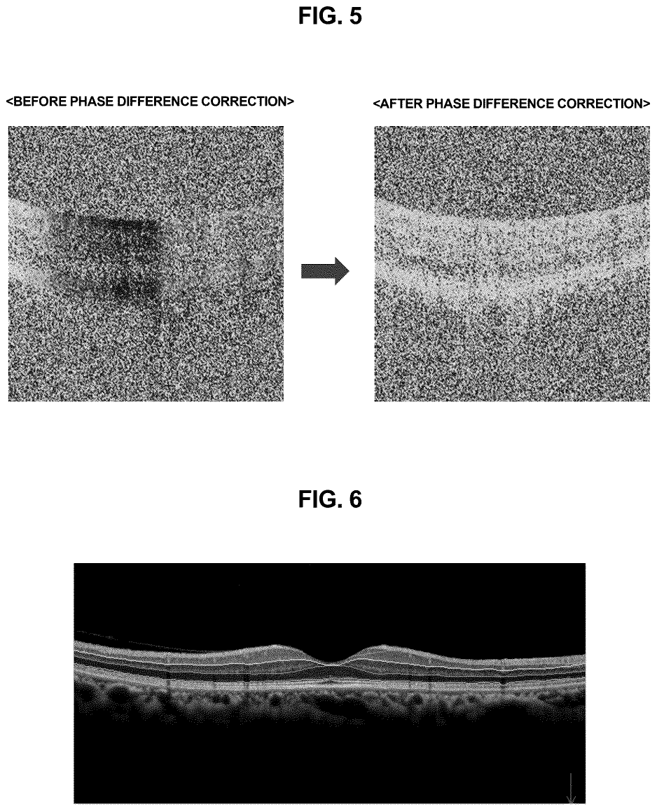

The CPU 31 corrects a phase difference of the first complex OCT signal Cn and a phase difference of the second complex OCT signal Cn+1 (S4). Ideally, the phase difference should be small in a region where no blood vessel is present (a non-vascular region). However, in practice, even in a region where no blood vessel is present, due to fluctuations of the living body and the like, the phase difference between A-scan lines within each B-scan image caused by each complex OCT signal often becomes large. In this case, even if alignment between the first complex OCT signal Cn and the second complex OCT signal Cn+1 is performed in S3, the quality of the generated motion contrast data decreases. In contrast, by reducing the phase difference in the non-vascular region through the processing of S4, motion contrast data of higher quality is more easily generated. A specific method for the processing of S4 can be selected as appropriate. For example, the CPU 31 may calculate a complex conjugate product at each pixel of an A-scan, accumulate the calculated products, and calculate an argument. The calculated argument is a phase difference of the entire A-scan. The CPU 31 may correct the phase difference by shifting the OCT complex signal at time t by the calculated phase difference. FIG. 5 shows an example of a comparison result between a B-scan image before phase-difference correction and a B-scan image after phase-difference correction.

The CPU 31 executes segmentation processing for detecting at least one of a layer and a boundary of a biological tissue (in the present embodiment, layers and boundaries of fundus tissue) from at least a portion of a plurality of images aligned in S3 (a B-scan image generated by the first complex OCT signal Cn and a B-scan image generated by the second complex OCT signal Cn+1) (S5). The result of the segmentation processing acquired in S5 is used, for example, in processing of S8 described below to generate an Enface image of one or more specific layers. FIG. 6 shows an example of a result obtained by detecting a plurality of layers and boundaries from a B-scan image.

As an example, in the present embodiment, a mathematical model trained by a machine-learning algorithm is used. The mathematical model is trained in advance using a plurality of training data items including B-scan images so as to output, upon input of a B-scan image, a detection result of layers and boundaries appearing in the input B-scan image. The CPU 31 obtains a detection result of layers and boundaries in a B-scan image by inputting the B-scan image to the mathematical model. However, as a method of the segmentation processing, it is also possible to use another method (for example, a method using known image processing, etc.). In S5, the CPU 31 may detect layers and boundaries of an averaged-addition image obtained by adding (which may be addition averaging) a plurality of images aligned in S3. In this case, noise of the B-scan image to be subjected to detection of layers and boundaries is reduced as compared with noise of the B-scan images before being added. Therefore, layers and boundaries are more easily detected with higher accuracy.

The CPU 31 calculates an amount of change between a first complex OCT signal Cn and a second complex OCT signal Cn+1 (S6). The amount of change calculated in S6 is not an amount of change in only the amplitude of the first complex OCT signal Cn and the second complex OCT signal Cn+1, nor is it an amount of change in only the phase of the first complex OCT signal Cn and the second complex OCT signal Cn+1. That is, the amount of change calculated in S6 is an amount of change between two complex OCT signals. Therefore, the amount of change calculated in S6 reflects both amplitude information and phase information in the two complex OCT signals.

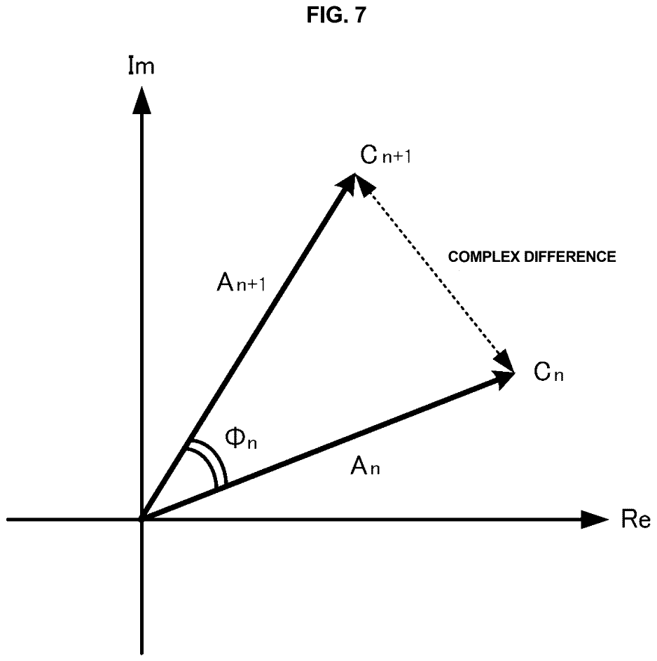

As an example, in S6 of the present embodiment, the CPU 31 calculates a complex difference |Cn−Cn+1| between the first complex OCT signal Cn and the second complex OCT signal Cn+1 as an amount of change between the two complex OCT signals. As shown in FIG. 7, a complex OCT signal can be represented on a complex plane in which one axis (horizontal axis Re) is a real axis and the other axis (vertical axis Im) is an imaginary axis. As shown in FIG. 7, the complex difference between the first complex OCT signal Cn and the second complex OCT signal Cn+1 reflects both amplitude information and phase information in the two complex OCT signals. “φn” is a phase difference of the first complex OCT signal Cn and the second complex OCT signal Cn+1. The complex difference can be calculated by the following Equation (1). However, instead of the complex difference itself, a result obtained by performing some further calculation using the complex difference may be used as an amount of change between the two complex OCT signals.

$$\vert {C}_{n} - {C}_{n + 1} \vert = \sqrt{{A}_{n}^{2} + {A}_{n + 1}^{2} - 2 {A}_{n} {A}_{n + 1} cos {∅}_{n}} \qquad [ Equation 1 ]$$

Here, it is also possible to generate motion contrast data using an amount of change (in the present embodiment, a complex difference) between the first complex OCT signal Cn and the second complex OCT signal Cn+1 calculated in S6. However, since the amount of change calculated in S6 is not normalized by the magnitude of the signal, an artifact tends to appear in a high-luminance region even when there is actually no motion of the subject.

Accordingly, the CPU 31 normalizes the amount of change calculated in S6 based on the magnitude of the first complex OCT signal Cn and the second complex OCT signal Cn+1, and generates motion contrast data using the normalized value (S7). As a result, the amount of change calculated in S6 is normalized while maintaining influences of both phase information and amplitude information, and motion contrast data is generated. Note that, by repeatedly executing the processing of S1 to S7 for each of a plurality of scanning lines 58 (see FIG. 2), motion contrast data for the entirety of the two-dimensional measurement region 55 is generated.

An example of specific processing in S7 will be described. The complex difference represented by (Equation 1) takes the maximum value represented by (Equation 2) when “φn=±π”.

$$\sqrt{{A}_{n}^{2} + {A}_{n + 1}^{2} - 2 {A}_{n} {A}_{n + 1} cos {∅}_{n}} = \sqrt{{( {A}_{n} + {A}_{n + 1} )}^{2}} = {A}_{n} + {A}_{n + 1} \qquad [ Equation 2 ]$$

“An+An+1=|Cn|+|Cn+1|” is given. Therefore, as shown in (Equation 3), by dividing the complex difference calculated in S6 by “|Cn|+|Cn+1|”, it can be normalized such that the maximum value becomes “1”. However, the processing method in S7 may be changed. For example, the normalization method in S7 may be appropriately changed in accordance with the method of calculating the amount of change in S6.

$$\frac{\vert {C}_{n} - {C}_{n + 1} \vert}{\vert {C}_{n} \vert + \vert {C}_{n + 1} \vert} \qquad [ Equation 3 ]$$

Further, the CPU 31 applies smoothing processing, noise removal processing, and the like to the motion contrast data generated by the processing in S6 and S7. For example, at least one of a Gaussian filter, a box filter, a bilateral filter, and the like may be applied to the motion contrast data. Further, for example, after applying various filters to each of the numerator and the denominator of (Equation 3), the numerator may be divided by the denominator.

The CPU 31 generates Enface images (angiography images) of one or more specified layers on the basis of the segmentation result acquired in S5 (S8). As an example, in the processing of S8, the CPU 31 may generate a tomographic vascular image based on motion contrast data generated for each scan line 58. The CPU 31 may generate an Enface image of a specified layer by identifying the specified layer from the tomographic vascular image based on the segmentation result and accumulating the pixel values of the identified specified layer in the Z direction, or by taking the maximum value of the pixel values of the specified layer in the Z direction. Needless to say, the CPU 31 may generate Enface images for all layers in the fundus.

### (Description of Acquired Motion Contrast Data)

The characteristics of motion contrast data (data based on a normalized complex difference) generated by the OCT signal processing according to the present embodiment will be described. First, the technical background will be described with reference to FIG. 9. As illustrated in FIG. 9 (A), when a plurality of complex OCT signals are acquired at a non-vascular location, the amplitude of each signal tends to become large, the change in amplitude between the plurality of signals tends to become small, and the change in phase between the plurality of signals also tends to become small. As illustrated in FIG. 9 (B), when a plurality of complex OCT signals are acquired at a vascular location, the amplitude of each signal, the change in amplitude between the plurality of signals, and the change in phase between the plurality of signals all tend to become large. Further, as illustrated in FIG. 9 (C), focusing on noise of each of a plurality of OCT signals acquired from the same location at different times, the amplitude of each noise tends to become small, whereas both the change in amplitude between the plurality of noises and the change in phase between the plurality of noises tend to become large. As described above, both amplitude characteristics and phase characteristics differ among signals at non-vascular locations, signals at vascular locations, and noise. Therefore, if motion contrast data is generated using only information of one of amplitude and phase, it tends to become difficult to appropriately distinguish signals at non-vascular locations, signals at vascular locations, and noise.

In contrast, according to the OCT signal processing of the present embodiment, the amount of change calculated in S6 is normalized in S7 while maintaining the effects of both phase information and amplitude information, thereby generating motion contrast data. That is, motion contrast data reflecting both phase information and amplitude information is generated. Accordingly, the quality of the motion contrast data is improved as compared with a case where only one of amplitude information and phase information is used.

In addition, the amount of change between the first complex OCT signal Cn and the second complex OCT signal Cn+1 (in the present embodiment, the amount of change calculated by the processing of S6) is susceptible to the influence of the sensitivity of the complex OCT signal. Therefore, if the amount of change between the plurality of signals is used for generating the motion contrast data without being normalized, a strong signal may appear in the motion contrast data as an artifact in a region having high luminance (a high-luminance region) even when there is actually no motion of the subject. In this case, it becomes difficult to distinguish an artifact in the high-luminance region from an actual vascular region. Thus, it is assumed that first motion contrast data generated based on an amount of change between a plurality of OCT signals is multiplied by second motion contrast data that does not depend on the magnitude of the OCT signal. In this case, the sensitivity of the first motion contrast data is high. On the other hand, the sensitivity of the second motion contrast data inevitably becomes low. Therefore, when the two sets of data are multiplied, although artifacts in the high-luminance region may be reduced, the sensitivity decreases overall. As a result, the likelihood increases that vascular regions that are actually continuous will be interrupted.

In contrast, according to the OCT signal processing of the present embodiment, the amount of change calculated in S6 is appropriately normalized in S7 while high sensitivity is maintained, thereby generating motion contrast data. Therefore, better motion contrast data is more likely to be generated.

In addition, a method is also conceivable in which each of the first complex OCT signal Cn and the second complex OCT signal Cn+1 is first normalized by the signal magnitude, and thereafter an amount of change in the magnitude-normalized values is calculated. However, if each of the two complex OCT signals is normalized by magnitude before calculating the amount of change, amplitude information is lost at that point, and motion contrast data based only on phase information is generated. Motion contrast data based only on phase information is prone to noise. In particular, when generating a motion contrast image of a still image, it is more desirable to use both amplitude information and phase information. In contrast, according to the OCT signal processing of the present embodiment, motion contrast data is generated that reflects both phase information and amplitude information and also has high sensitivity.

In addition, a technique (hereinafter, referred to as “technique Ar”) is also known in which the length of an arc formed on the complex plane by the first complex OCT signal Cn and the second complex OCT signal Cn+1 is calculated based on Equation (4), and motion contrast data is generated based on the calculated result.

$$\frac{{( {A}_{n} + {A}_{n + 1} )}^{2} {∅}_{n}^{2}}{{A}_{n}^{2} + {A}_{n + 1}^{2}} \qquad [ Equation 4 ]$$

In Equation (4), both the amplitude and the phase difference are used. However, in technique Ar, since the phase difference φn is multiplied as is to generate the motion contrast data, motion contrast data that are overly sensitive to the phase difference are generated. In particular, according to technique Ar, when the phase difference is small, the motion contrast value becomes a small value even if the amplitude difference is very large, and therefore, in some cases, good motion contrast data cannot be obtained.

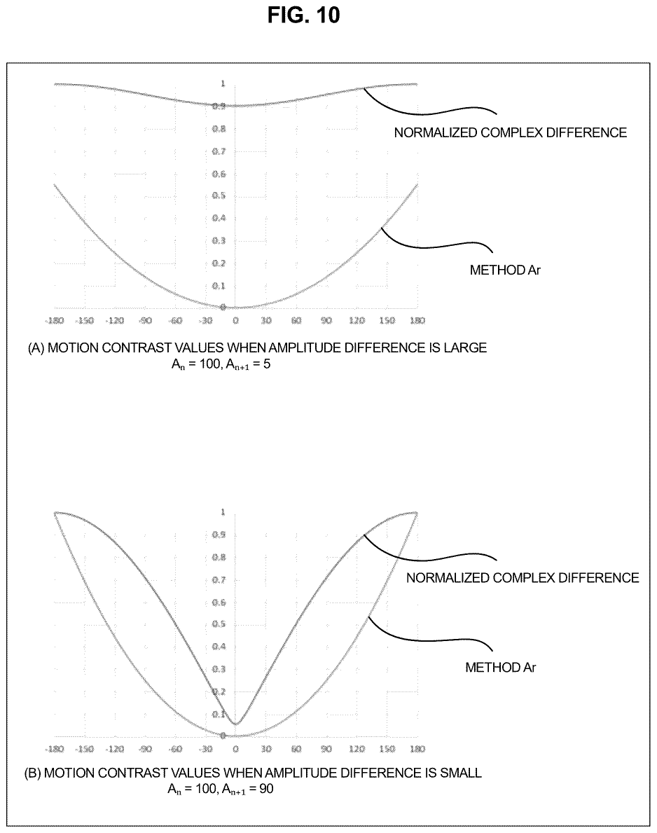

FIG. 10 is a graph comparing changes in the values of motion contrast data (data based on the normalized complex difference) generated by the OCT signal processing according to the present embodiment and motion contrast data according to technique Ar, when the phase difference is varied with the amplitude value fixed. In FIGS. 10 (A) and 10 (B), the vertical axis indicates the value of the normalized data, and the horizontal axis indicates the phase difference. As shown in FIG. 10, in the data according to technique Ar, when the value in the case where the amplitude difference is large (the case of FIG. 10 (A)) and the value in the case where the amplitude difference is small (the case of FIG. 10 (B)) are compared with each other at the same phase difference, the value of FIG. 10 (B), in which the amplitude difference is small, becomes conversely larger than the value of FIG. 10 (A), in which the amplitude difference is large. This property is undesirable as a property of motion contrast data for imaging changes in a signal. Furthermore, in the data according to technique Ar, in both the case where the amplitude difference is large (the case of FIG. 10 (A)) and the case where the amplitude difference is small (the case of FIG. 10 (B)), the data value is “0” when the phase difference is “0”. In other words, in the data according to technique Ar, when the phase difference becomes “0”, the value becomes “0” regardless of the amplitude difference. As described above, the data according to technique Ar are overly sensitive to the phase difference.

In contrast, focusing on the data based on the normalized complex difference, when the amplitude difference is large (the case of FIG. 10 (A)), the value is always large regardless of the phase difference, and the value gently increases and decreases in accordance with an increase and decrease in the phase difference. Further, when the amplitude difference is small (the case of FIG. 10 (B)), the value greatly increases and decreases in accordance with an increase and decrease in the phase difference, and the value approaches “0” only when both the amplitude difference and the phase difference become small. As described above, the motion contrast data generated by the OCT signal processing according to the present embodiment are data in which both the amplitude difference and the phase difference are appropriately reflected.

The technique disclosed in the above embodiment is merely an example. Therefore, it is also possible to modify the technique exemplified in the above embodiment. For example, it is also possible to execute only a part of the plurality of techniques exemplified in the above embodiment. Further, in the processing of S6 and S7 of the above embodiment, normalization is performed such that the maximum value becomes 1 by dividing the complex difference |Cn−Cn+1| by |Cn|+|Cn+1|. However, normalization may also be performed such that the maximum value is 1 and the minimum value is 0. As an example, letting “m=|Cn|+|Cn+1|” and “M=|Cn|+|Cn+1|”, normalization may be performed such that the maximum value is 1 and the minimum value is 0 by calculating the following Equation (5). Further, a process of calculating at least one of a square of the complex OCT signal, a square of the complex difference, and the like may be included in the process of generating the motion contrast data.

$$\frac{\vert {C}_{n} - {C}_{n + 1} \vert - m}{M - m} \qquad [ Equation 5 ]$$

The process of calculating an amount of change between a plurality of complex OCT signals in S6 of FIG. 4 is an example of a “change-amount calculation step.” The process of normalizing the amount of change in S7 is an example of a “normalization step.” The process of correcting a phase difference in S4 is an example of a “phase-difference correction step.”

1. An OCT apparatus, comprising:
an OCT unit configured to detect an OCT signal based on reference light and measurement light irradiated onto biological tissue of a subject; and
a control unit configured to generate motion contrast data in the biological tissue by processing the OCT signal, wherein
the control unit is further configured to perform:
a change-amount calculation step of calculating an amount of change between a first complex OCT signal Cn and a second complex OCT signal Cn+1 that were acquired at different times at a same position on the biological tissue; and
a normalization step of generating the motion contrast data by normalizing the calculated amount of change based on a magnitude of the first complex OCT signal Cn and the second complex OCT signal Cn+1.

2. The OCT apparatus according to claim 1, wherein
at the change-amount calculation step, the control unit is further configured to calculate, as the amount of change, a complex difference |Cn−Cn+1| between the first complex OCT signal Cn and the second complex OCT signal Cn+1.

3. The OCT apparatus according to claim 1, wherein
the control unit is further configured to, at the normalization step, normalize the amount of change by using, as the magnitude of the first complex OCT signal Cn and the first complex OCT signal Cn+1, a sum |Cn|+|Cn+1| of an absolute value |Cn| of the first complex OCT signal Cn and an absolute value |Cn+1| of the second complex OCT signal Cn+1.

4. The OCT apparatus according to claim 1, wherein
the control unit is further configured to perform:
a phase difference correction step of correcting a phase difference of the first complex OCT signal Cn and a phase difference of the second complex OCT signal Cn+1; and
the change-amount calculation step and the normalization step with respect to the first complex OCT signal Cn whose phase difference has been corrected and the second complex OCT signal Cn+1 whose phase difference has been corrected.

5. A non-transitory, computer readable, storage medium storing an OCT signal processing program executed by an OCT signal processing apparatus that processes an OCT signal obtained by reference light and measurement light irradiated onto a biological tissue of a subject, the OCT signal processing program, when executed by a control unit of the OCT signal processing apparatus, causing the OCT signal processing apparatus to perform:
a change-amount calculation step of calculating an amount of change between a first complex OCT signal Cn and a second complex OCT signal Cn+1 that were acquired at different times at a same position on the biological tissue; and
a normalization step of generating motion contrast data by normalizing the calculated amount of change based on a magnitude of the first complex OCT signal Cn and the second complex OCT signal Cn+1.

6. An OCT signal processing method implemented by an OCT signal processing apparatus that processes an OCT signal obtained by reference light and measurement light irradiated onto a biological tissue of a subject, the OCT signal processing method comprising:
a change-amount calculation step of calculating an amount of change between a first complex OCT signal Cn and a second complex OCT signal Cn+1 that were acquired at different times at a same position on the biological tissue; and
a normalization step of generating motion contrast data by normalizing the calculated amount of change based on a magnitude of the first complex OCT signal Cn and the second complex OCT signal Cn+1.
